(19) United States
(12) Patent Application Publication
(10) Pub. No.: US 2010/0161050 A1
(43) Pub. Date: Jun. 24, 2010

(54) ACCOMMODATIVE INTRAOCULAR LENS

(76) Inventors: Ulfert Detmers, Hage (DE); Matthias Meyer, Nortmoor (DE)

Correspondence Address: Muncy, Geissler, Olds & Lowe, PLLC, 4000 Legato Road, Suite 310, FAIRFAX, VA, 22033, US

(21) Appl. No.: 12/528,423

(22) PCT Filed: Aug. 1, 2007
(86) PCT No.: PCT/EP07/06802

§ 371 Date: Oct. 1, 2009

(30) Foreign Application Priority Data
Feb. 25, 2007 (DE) 10 2007 009 502.5

(51) Int. Cl. A61F 2/16

(52) U.S. Cl. 623/6.13

(57) ABSTRACT

A phaco intraocular lens has a block made of optically transparent lens material with a first two-dimensional lens part and an opposite second two-dimensional lens part, wherein the first lens part is designed for coming into contact with the natural lens of the human eye. The first lens part has in this case greater deformability than the second lens part, a cavity being formed in the block between the first lens part and the second lens part.

The invention relates to an accommodative intraocular lens for phaco, i.e. natural lens-containing eyes according to the preamble of claim 1.

For the correction of refraction errors of the human eye, the prior art discloses artificial lenses which are added to the natural eye lens by implantation. In the customary methods, the implantation of such an intraocular lens is effected as an artificial lens in addition to the natural, biological eye lens. The artificial lens is intended to compensate a refraction error and replace spectacles or make them more compatible, the implantation usually being effected via a corneal incision with foldable or rigid artificial lenses.

In the methods of the prior art, two implantation sites are usually chosen:
a.) in front of the iris as a so-called anterior chamber lens
b.) between iris and natural lens as a so-called posterior chamber lens

Lenses to date which have been used for implantation in eyes with a natural lens consist of silicone, hydrogel or rigid as well as flexible acrylates. These materials have proved their worth many times. Although it has been possible to date to compensate a refraction error therewith, the phaco lenses cannot correct the accommodation difficulty increasing with age (presbyopia), so that once again an additional correction is required, for example by spectacles.

An object of the invention is to provide an improved intraocular artificial lens.

A further object is to provide an intraocular artificial lens which, when implanted in the eye, both reduces the lack of accommodation due to age to a considerable extent and compensates optical refraction errors.

These objects are achieved by the subjects of claim 1 or of the dependent claims or the solutions are further developed.

The invention is based on the formation of an intraocular lens in an accommodative, in particular pneumatic, manner, resulting in an artificial lens which can be added to the natural eye lens and, when implanted surgically in the eye, both reduces the lack of accommodation due to age to a considerable extent and compensates optical refraction errors and thereby permits a high degree of visual acuity without applicable aids, such as spectacles, for as long as possible.

According to the invention, the accommodative capacity of the eye which is still present in advanced age is utilized, the effectiveness of the reduced accommodation in advanced age being increased several times over by the optical and mechanical properties, according to the invention, of the artificial lens.

The effect of an improved action of the residual accommodation present in advanced age is achieved by virtue of the fact that a flexible artificial lens having a refractive index close to 1, which is surrounded by optically active media having greater refractive indices, for example of about 1.3 to 1.4, is brought into a concave shape by the accommodative movement or deflection of the natural lens. The total refractive power of the eye is therefore increased thereby, in that an artificial medium having a very low refractive index undergoes a change in shape driven by the accommodative movement of the natural lens, is forced into an increasingly concave shape and thus increases the total refractive power of the eye, permitting effective close-range focusing.

In principle, the difference between the refractive indices of the individual optical elements determines the magnitude of the total refractive power of an optical system. For the natural lens in contact with the surrounding aqueous humor, the difference between the refractive indices is about 0.07. The difference in the refractive indices between a flexible liquid or gaseous lens is up to 0.386, depending on the material. Accordingly, an accommodative lens curvature which results in only a change of refractive power of 0.5 diopter in the case of a small refractive index difference of 0.053 will be several times more efficient in the case of an index difference of 0.386.

The phaco intraocular lens according to the invention is described or illustrated in more detail below, purely by way of example, with reference to a posterior chamber lens.

Such an intraocular lens as a posterior chamber lens can be implanted between the natural biological lens and the iris. That side of the artificial lens which faces the lens then consists of a thin layer of the artificial lens carrier material. Enclosed in the artificial lens is a cavity which may also be filled with gas, liquid, oil or a solid, ideally with a gas for the mode of operation of this artificial lens according to the invention. The side facing the iris consists of a thicker layer of the artificial lens carrier material or of another material. In the peripheral region of the artificial lens, possibilities for volume compensation of the cavity filling are created in order to enable the interior lens to be effective. In the case of a gaseous design of the interior space of the artificial lens, atmospheric pressure variations are compensated thereby. As a result of this design, the radius of the outside remains substantially stable and the interior lens deforms in the case of an accommodation-related decrease in the radius of the inside of the artificial lens.

Thus, the artificial lens according to the invention is composed of three functionally different components of sections which are preferably formed from biologically inert materials which do not react chemically with one another and are completely diffusion-tight for the substances of the artificial lens and are optically transparent:
A flexible component which, separated by a liquid film, rests against the natural lens so that accommodative movements of the natural lens are reproduced by this flexible component directly and as far as possible to the full extent. A hydrophilic surface design of the flexible component can be effected in order to ensure a stable liquid layer between natural lens and artificial lens. The shape of the flexible component is advantageously configured so that the thickness increases continuously from the center to the periphery.
An intermediate component having a refractive index which is less than that of the surrounding optical media (natural lens, aqueous humor). This component rests directly against the flexible component and follows the movements of the flexible component to the full extent. For the purpose of amplifying the refractive power in the case of low residual accommodation, media having a low, in particular very low refractive index are required. These conditions are preferably fulfilled by gases and some liquids. According to the invention, gases or fluid media which are not capable of diffusing through the lens materials used are preferably employed. The requirement for biologically inert gases or liquids can be limited in this area in that those substances which are not inert but are completely safe can also be used. In principle, all low-boiling substances which are converted into the gaseous state at the physiological temperatures and pressures within the human eye and which are biologically inert but at least chemically inert for the lens material used are suitable, it being necessary to ensure diffusion impermeability of the lens material. These include, for example,
   carbon dioxide,
   noble gases, i.e. argon, neon, krypton, xenon, and
   sulfur hexafluoride,
   octafluoropentane,
   perfluorobutane,
   perfluoropentane,
   octafluorocyclopentane,
   perfluorocyclopentane,
   perfluoromethylcyclopentane,
   perfluorocyclohexane,
   hydrofluoroether,
   perfluoroketone or
   perfluorocyclohexane.
Although some of these substances are toxic, they can be used, at least theoretically, if the enclosing materials are sufficiently impermeable.
A rigid or comparatively inflexible component. This component encloses the gaseous or liquid component by a completely tight circular connection to the flexible component in the periphery. The peripheral thickening of the flexible component is compensated in the case of the inflexible component by a central thickening. Together with the flexible component, this forms the shape of the gaseous or liquid component. The inflexible component is furthermore suitable for making corrections for visual defects. By combining firm and flexible components, the result is a gap-like space between the two, which contains the optically active zone for accommodation amplification by changing the shape of the gaseous or liquid component.

In the case of distance adjustment of the eye, the gaseous or liquid component, which is referred to below as gas lens, is optically effective, i.e. the interfaces of flexible and inflexible component are expediently parallel to one another.

As a result of an accommodative movement of the natural lens, the reduced radius of curvature thereof is applied to the radius of curvature of the flexible component, and the gas lens thus subsequently acquires a concave characteristic. The change in shape of the gas lens would in certain circumstances lead to a pressure increase within the gas lens without compensation mechanisms, with the result that the application of the changes in the radii of curvature of the natural lens to the gas lens would be hindered since pressure differences would have to be overcome.

Two fundamentally different mechanisms are possible for pressure compensation. Firstly, the excess amount of gas or liquid can be removed from the gap to the periphery through peripheral openings into a corresponding space likewise located at the edge, where said amount of gas or liquid is then available for recycling as required, or a deflection of the inflexible component in the direction of the iris is permitted by a flexible connection between flexible component and inflexible component, with the result that pressure relief is ensured. Owing to the very small volumes, these compensating movements are unproblematic in view of the spatial relationships which exist within the eye. Since a certain hydrostatic pressure gradient exists, depending on position, the inflexible component is not shifted equally far in the direction of the iris at all points in the case of accommodation, so that a slight prismatic effect may result. Since this effect occurs on both sides and in the same direction, it cannot be registered in the case of a binocular visual process.

On removal of the displaced amounts of gas or liquid to peripheral membrane spaces of the artificial lens, constant positioning of the inflexible component relative to the flexible component is possible but the manufacturing steps for the production of such a lens are considerably more complicated. Since the pressure equilibration is effected through peripheral openings, the equilibration can, if appropriate, be manipulated externally by, for example, use of laser radiation and the extent can, if appropriate, thus be varied.

In order to apply the inflexible component in a moveable manner, the periphery of the flexible component can be designed with a double layer, which can be ensured, for example, by the larger peripheral thickness. The two layers coalesce toward the center. The inflexible component is fastened to the layer facing the iris. On pressure compensation, the two layers are lifted off one another or approach one another again. The double-layer nature of the periphery of the flexible component can be established comparatively easily during production on a lathe-like device by an incision whose edges are rounded.

When the artificial lens is designed with gas, in particular the diffusion behavior has to be taken into account. Here, correspondingly diffusion-tight materials (e.g. hydrophobic acrylates) can be used as well as additionally or alternatively the abovementioned gases which do not diffuse or diffuse only extremely slowly, such as, for example, noble gases having large atoms or molecular compounds. Gases which form under physiological conditions and may diffuse into the lens must be introduced during the production of the lens itself in such concentrations that they are in diffusion equilibrium at the implantation site with the gases occurring there.

Since the edge zones of the artificial lens have reflective structures, reflection-suppressing or reflection-reducing measures, such as, for example, the blackening of the lens material, can be carried out. For avoiding troublesome light reflections, the material can be provided with a light-proof treatment in the region of the compensating spaces in the peripheral region.

The artificial lens according to the invention is described in more detail below, purely by way of example, with reference to working examples shown schematically in the drawing. Specifically,

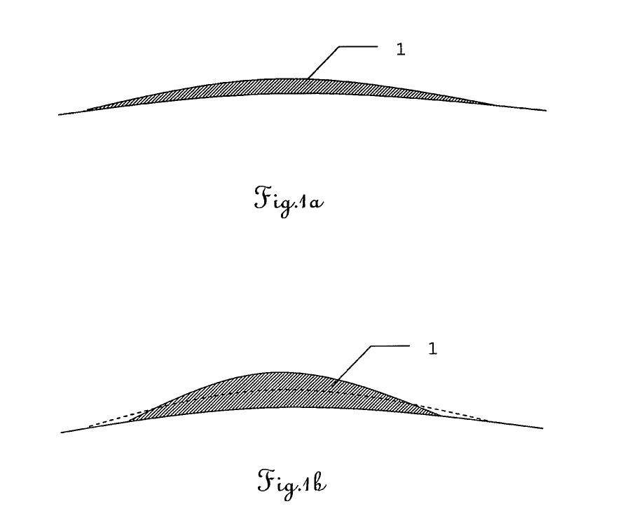

FIG. 1a-b show the schematic diagram of the accommodation of a natural lens;

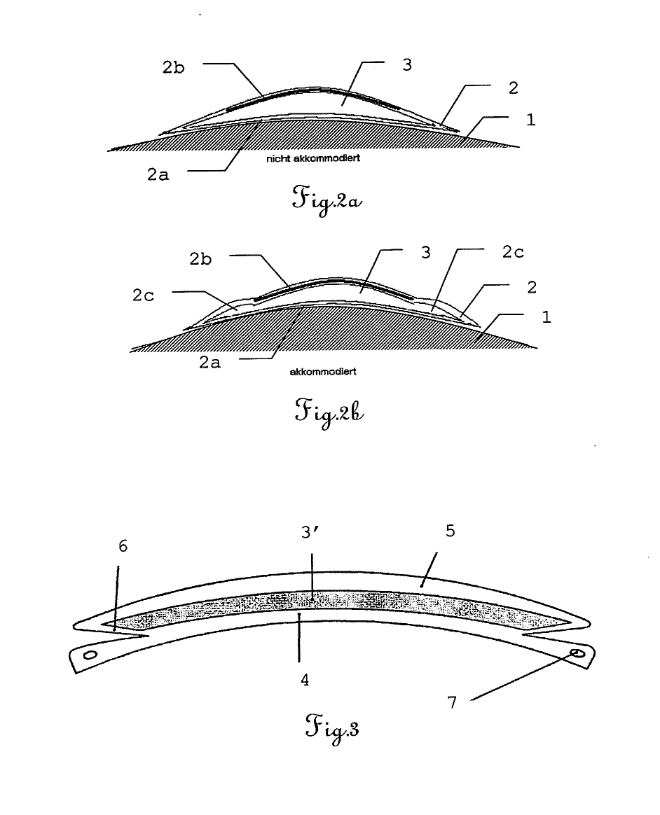

FIG. 2a-b show the schematic diagram of the accommodation of a first embodiment of the lens according to the invention;

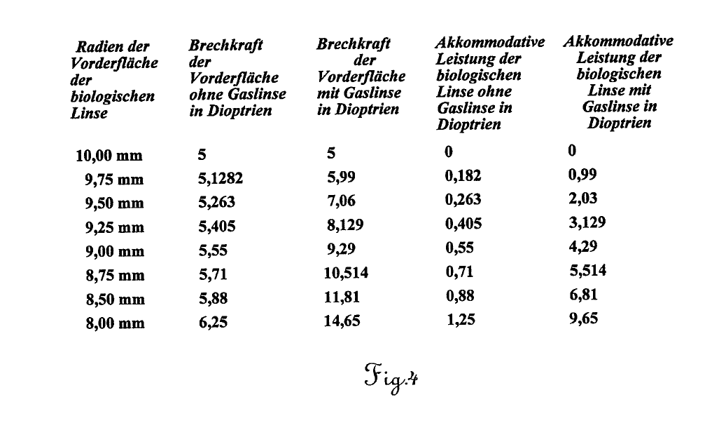

FIG. 3 shows the schematic diagram of a second embodiment of the lens according to the invention and

FIG. 4 shows the influence of a variable gas lens on the refractive power of the biological lens in the interior of the eye.

FIG. 1a-b show the schematic diagram of the accommodation of a natural lens 1, FIG. 1a showing the relaxed, non-accommodated rest state of the lens 1 and FIG. 1b showing the accommodated state.

FIG. 2a-b show the schematic diagram of the accommodation of a first embodiment of an accommodative artificial or intraocular lens according to the invention, comprising a one-piece lens body 2 and a cavity 3 incorporated therein and intended for amplifying the refractive effect of the accommodative residual power of the natural lens 1. The lens body 2 has a first laminar lens part 2a and a second laminar lens part 2b, the first lens part 2a being formed for direct or indirect contacting of the natural lens of the human eye and being opposite the outward-oriented second lens part 2b. FIG. 2a shows the relaxed, non-accommodated rest state of the artificial lens and FIG. 2b shows the accommodated state. The front and back surfaces of the cavity 3 may also be formed in such a way that they are parallel to one another in the non-accommodated state, i.e. without externally produced deformation, the first lens part 2a having the smallest thickness in the central region and the second lens part 2b having its greatest thickness in the central region.

For this purpose, it is necessary for the artificial lens to be substantially adapted to the front curvature of the natural lens 1 in order to follow the contour of this as far as possible in the same direction and to make contact over the whole area. If the change in curvature of the front lens surface of the natural lens 1, shown in FIG. 1b, occurs, the back surface of the artificial lens, i.e. the first lens part 2a, experiences the same changes, the artificial lens floating on a thin liquid film on the natural eye lens. By corresponding design, for example by means of a coating with a material having increased refractive index, the front surface of the artificial lens, i.e. the second lens part 2b, experiences a stronger refractive power, and the increase in refractive power of the natural lens 1 is amplified. As a result of this, the residual accommodative capacity obtained is increased so that a reading capacity lost or endangered in the meantime can be regained. The first lens part 2a may also have a hydrophilic coating for stabilizing a liquid layer between artificial lens and natural lens 1.

Here, the carrier material of the lens body 2 may consist of conventional transparent plastics, as have been customary to date in surgical ophthalmology (silicone, acrylates, hydrogel, etc). By formation of different material thicknesses for first lens part 2a and second lens part 2b, it is possible to ensure that the thicker second lens part 2b is deformed by the residual accommodation of the eye to a lesser extent than the first lens part 2a which is kept comparatively thinner. For achieving high mechanical stability, the peripheral regions of first and/or second lens part 2a, 2b can be formed to be thicker than the corresponding central regions, in particular with a continuous and stepless transition.

The additional accommodative effect also results from a cavity configuration of the artificial lens, the cavity 3 preferably being filled with transparent liquid, transparent gas or a transparent and deformable solid having inert properties and optical purity. The effect of the improved action of the residual accommodation present in advanced age is achieved by virtue of the fact that the—for example gas-filled—cavity 3 may be considered as a further lens enclosed in the carrier lens and is brought into a more concave shape by the accommodative movement or deflection. The cavity 3 is formed in such a way that, in the case of a change of radius of the first lens part 2a by accommodative movements of the natural lens 1 in the eye, a comparatively smaller change of radius of the second lens part 2b occurs so that a change in the concavity of the cavity 3 results.

In order to achieve an increase in the total refractive power of the eye thereby, the optical material of the inner liquid or gas lens should be chosen so that its refractive index is lower than that of the natural lens and of the surrounding aqueous humor. This requirement is met by gases and some liquids.

In the case of an appropriate design of the intraocular lens, pockets arranged at the edge or peripheral compensation volumes 2c for volume compensation of the cavity filling during the deformation of the total structure form in the accommodated state. As a result, the internal pressure can be kept constant so that a pressure compensation is brought about. The lens material can be chosen so that a filling gas in the cavity 3 as a gas lens does not diffuse through the lens material. A further point to be taken into account when choosing the lens material is the permeability for other gases which occur in a human body in dissolved form or in a form attached to blood constituents, such as, in particular, oxygen or nitrogen. It is advantageous here to choose a lens material permeable for these gases, for pressure/volume compensations by inward and outward diffusion.

FIG. 3 shows a second embodiment of the lens according to the invention with assembled structure in a schematic diagram. In this case, the structure is not integral but has a flexible membrane 4 as a component in contact with the natural lens of the eye and hence as a first lens part. Arranged opposite it is a comparatively inflexible front plate 5 as a second lens part, the components being fixed by a flexible connection 6. This rigid front plate 5 also permits, for example, the incorporation of optical corrections for compensating refraction errors of the eye.

Once again, a cavity 3′ which may have a gas or air filling is formed in the interior of the lens. For increasing the mechanical stability, the artificial lens may have a reinforcing structure 7, for example in the form of a hollow, all-round segmented ring. Ballast weights, in particular annular weights in the edge zones, may also be housed in the periphery in a similar manner, for compensation of buoyancy effects relating to a gas filling. If these ballast weights are interlinked with one another by a mechanically stabilizing flexible connection, stabilization of the total lens body is effected.

The influence of a variable gas lens on the refractive power of the biological lens in the interior of the eye is illustrated in FIG. 4 with reference to the results of an exemplary calculation. The fundamental calculation assumes that a flexible gaseous lens is positioned between iris and biological lens. This gaseous lens is enclosed in the flexible membrane and varied in its refractive power. The change in the refractive power of the gas lens is achieved by utilizing the amplification of the curvature of the biological lens on accommodation to bring the gas lens into a concave shape. That side of the gas lens which faces the iris remains constant here. A normal-sighted eye which requires no optical correction at all for distance vision was assumed for the lens calculations. Accordingly, the front surface and back surface of the gas lens are parallel to one another so that there is no optical effect in the sense of a collecting or dispersing effect (distance accommodation). Likewise, the optical parameters of the flexible and of the rigid part of the artificial lens are dimensioned so that there is no collecting or dispersing effect in totality, i.e. the functioning of the artificial lens is based only on the change in shape of the gas lens.

Since therefore the flexible part and the rigid part of the artificial lens have no collecting or dispersing effect, these contributions were not taken into account in the lens calculation. Although the thickness of the artificial lens makes a certain contribution to the overall refraction, this effect is several times smaller than the overall effect of the artificial lens in the case of a change in shape of the gas lens, so that the principle of the artificial lens is not decisively affected thereby. A knowledge of the associated refractive indices is required only for calculating the effect of the thickness of the artificial lens. This means in the end that a simplified model is used, without taking into account the thickness of the artificial lens.

Regarding the materials, it should be stated in principle that primarily flexible and, if appropriate, inflexible substances customary in eye surgery, such as, for example, acrylates or silicone compounds, can be used. For the inflexible part of the artificial lens, it is true that said part need only be functionally inflexible, i.e. it is entirely possible to use a flexible material provided that it fulfills the condition of shape stability in the case of an accommodating change of shape of the gas lens.

The expected amplification of the change in refractive power on accommodation is utilized in order to support the residual accommodation in advanced age in the case of presbyopia. The effect of the residual accommodation without and with gas lens, in diopters, is compared, the refractive power at the front end surface without a gas lens, i.e. under physiological conditions, starting from the state of distance accommodation (r=10 mm), first being calculated here. The radius of curvature of the front lens surface is reduced here in steps of 0.5 or 0.25 mm. In each case 8 calculations without and with gas lens are carried out.

Here, the calculation is based on the following relationships

$$\begin{matrix}f = \frac{{n}_{l} \cdot {r}_{l}}{{n}_{l} - {n}_{ah}} & ( 1 ) \\ D = \frac{{n}_{l}}{f} & ( 2 )\end{matrix}$$

which, after substitution, gives

$$D = \frac{{n}_{l} \cdot ( {n}_{l} - {n}_{ah} )}{{n}_{l} \cdot {r}_{l}} \qquad ( 3 )$$

This expression describes the refractive effect of the biological lens in the aqueous humor at its front surface. If it is intended to determine the refractive effect of the biological lens at its front surface together with the preceding gas lens, the following relationships result therefrom:

$$\begin{matrix}D = \frac{{n}_{gl} \cdot ( {n}_{gl} - {n}_{ah} )}{{n}_{gl} \cdot {r}_{gl}} + \frac{{n}_{l} \cdot ( {n}_{l} - {n}_{gl} )}{{n}_{l} \cdot {r}_{l}} & ( 4 ) \\ D = \frac{( {n}_{gl} - {n}_{ah} )}{{r}_{gl}} + \frac{( {n}_{l} - {n}_{gl} )}{{r}_{l }} & ( 5 )\end{matrix}$$

The following designations are used here
f=focal distance (in this case the proportion of the front lens surface, based on the total focal distance)
D=diopters
nₐₕ=refractive index of the aqueous humor (1.336)
nₗ=refractive index of the biological lens (1.386)
n_{gl}=refractive index of the gas lens (1.0003)
r_{gl}=radius of the gas lens at the front surface toward the iris (10 mm)
rₗ=radius of the gas lens at the back surface or the radius of the biological lens at the front surface (max. 10 mm)

The results obtained are shown in the form of a table in FIG. 4 and make it clear that a gas- or liquid-filled lens according to the invention constitutes an effective accommodation aid.

1. A phaco intraocular lens, for presbyopia correction, comprising a lens body of optically transparent lens material, preferably of silicone or acrylate, comprising a first laminar lens part and an opposite second laminar lens part, the first lens part being formed for contacting the natural lens of the human eye, wherein the first lens part has a higher deformability than the second lens part and a cavity in the lens body is formed between the first lens part of the second lens part.

2. The intraocular lens according to claim 1, wherein the cavity is formed in such a way that, in the case of a change in the radius of curvature of the first lens part by accommodative movements of the natural lens in the eye, a comparatively smaller change or no change in the radius of curvature of the second lens part takes place, so that a change in the concavity of the cavity results.

3. The intraocular lens according to claim 1, wherein the cavity is filled with
a liquid,
a gas or
a deformable solid
an optically transparent medium, the medium having a lower refractive index than the first and/or the second lens part.

4. The intraocular lens according to claim 1, wherein corrections are incorporated into the second lens part for compensating refraction errors of the eye.

5. The intraocular lens according to claim 1, wherein front surface and back surface of the cavity are parallel to one another in the non-accommodated state, the first lens part having the smallest thickness in the central region and the second lens part having its greatest thickness in the central region.

6. The intraocular lens according to claim 1, wherein the lens body is integral and is formed with a first lens surface as first lens part and a second lens surface as second lens part.

7. The intraocular lens according to claim 1, wherein in that the lens has an assembled structure with a flexible membrane as first lens part and a functionally inflexible front plate as second lens part.

8. The intraocular lens according to claim 1, wherein the lens has compensation volumes for pressure compensation, in particular in the periphery of the cavity.

9. The intraocular lens according to claim 1, wherein peripheral regions of first and/or second lens part are formed so as to be thicker than the corresponding central regions, in particular with a continuous and stepless transition.

10. The intraocular lens according to claim 1, wherein in that the first lens part is formed in the periphery as a circular double layer in the form of a hollow, all-round ring.

11. The intraocular lens according to claim 1, wherein the peripheral regions of the lens body are formed so as to be reflection-reducing and/or light-impermeable, in particular by blackening.

12. The intraocular lens according to claim 1, wherein the first lens part has a hydrophilic coating for stabilizing a liquid layer between artificial lens and natural lens.

13. The intraocular lens according to claim 1, wherein the cavity is in the form of a gas lens having a filling gas which does not diffuse through the lens material.

14. The intraocular lens according to claim 13, wherein the filling gas is at least one of the following gases
carbon dioxide
noble gases, i.e. argon, neon, krypton, xenon, and
sulfur hexafluoride,
octafluoropentane,
perfluorobutane,
perfluoropentane,
octafluorocyclopentane,
perfluorocyclopentane,
perfluoromethylcyclopentane,
perfluorocyclohexane,
hydrofluoroether,
perfluoroketone or
perfluorocyclohexane.

15. The intraocular lens according to claim 1, wherein, for pressure/volume compensations by inward and outward diffusion, the lens material is permeable for atmospheric gases which occur in the human body in dissolved form or in a form attached to blood constituents, such as, in particular, oxygen or nitrogen.

16. The intraocular lens according to claim 1, wherein ballast weights, in particular annular weights, in the edge zones for compensation of buoyancy effects relating to a gas filling.

17. The intraocular lens according to claim 16, wherein the ballast weights are connected to one another by mechanically stabilizing fixation.
